(19) United States
(12) Patent Application Publication
(10) Pub. No.: US 2011/0245316 A1
(43) Pub. Date: Oct. 6, 2011

(54) PREVENTION OR DELAY OF ONSET OF ORAL MUCOSITIS

(75) Inventor: Israel RIOS, Menlo Park, CA (US)

(73) Assignee: SCICLONE PHARMACEUTICALS, INC., Foster City, CA (US)

(21) Appl. No.: 13/074,828

(22) Filed: Mar. 29, 2011

Related U.S. Application Data
(60) Provisional application No. 61/319,050, filed on Mar. 30, 2010.
(60) Provisional application No. 61/366,655, filed on Jul. 22, 2010.

(51) Int. Cl. A61K 31/405; A61P 1/02; A61P 1/04; A61P 31/10; A61P 31/04; A61P 31/00

(52) U.S. Cl. 514/419

(57) ABSTRACT

The present invention provides a method for preventing or delaying the onset of oral mucositis, including the onset of ulcerative or severe OM, in a patient receiving cancer therapy. The method comprises administering to the patient an effective regimen of γ-D-glutamyl-L-tryptophan (SCV-07) over the course of therapy. The regimen, which includes scheduled doses of SCV-07 with respect to radiation exposure and/or chemotherapy, is effective for preventing or delaying the onset of OM. In accordance with the invention, the patient is more able to complete the planned course of cancer therapy (including chemotherapy and/or radiation therapy), by maintaining a sufficient nutritional state, and by avoiding the significant pain and discomfort associated with OM.

### PRIORITY

This application claims priority to U.S. Provisional Application No. 61/319,050, filed Mar. 30, 2010, and to U.S. Provisional Application No. 61/366,655, filed Jul. 22, 2010, both of which are hereby incorporated by reference in their entirety.

### FIELD OF THE INVENTION

The present invention relates to the prevention and/or delay of onset of oral mucositis during radiation and/or chemotherapy.

### BACKGROUND

The National Cancer Institute estimates that 400,000 patients in the U.S. suffer from oral mucositis (OM) during cancer therapy. OM is a painful, debilitating and costly toxicity of many of the drug or radiation regimens used to treat cancer. OM is a condition in which the sensitive cells lining the mouth and throat are damaged by cancer treatments, such as chemotherapy and radiation therapy, and become painful mouth sores. Severe OM has been reported to occur in about 50% of patients who receive chemoradiation for the prevention of cancers of head and neck. Importantly, radiation to the head and neck, especially when it includes the tissues of the mouth, pharynx and hypopharynx, almost always results in significant ulcerative OM. Symptoms can include painful ulcers in the mouth and throat, redness and swelling of the gums, dryness and overall soreness in the mouth, and difficulty eating, swallowing, talking and drinking.

In addition to the symptoms of OM and its impact on quality of life, mucositis adversely affects a variety of other health and economic outcomes, such as a risk of serious infection, the need for parenteral nutrition, narcotic analgesia, and increased hospitalization and feeding-tube placement. The development of severe or ulcerative OM can also compromise the patient's ability to complete the planned course of cancer therapy.

Preventing, treating, or ameliorating oral mucositis is therefore a serious unmet clinical need.

### SUMMARY OF THE INVENTION

The present invention provides a method for preventing or delaying the onset of oral mucositis (OM), including the onset of ulcerative or severe OM, in a patient receiving radiation therapy and/or chemotherapy. The method comprises administering to the patient an effective regimen of γ-D-glutamyl-L-tryptophan (SCV-07) over the course of the therapy. The regimen, which includes scheduled doses of SCV-07 with respect to radiation exposure and/or chemotherapy administration, is effective for preventing or delaying the onset of OM. In accordance with the invention, the patient is more able to complete the planned course of therapy, by maintaining a sufficient nutritional state, and by avoiding the significant pain and discomfort associated with OM.

In certain embodiments, the patient receives radiation therapy for head and neck cancer. For example, the cancer may be a squamous cell carcinoma of the oral cavity, oropharynx, hypopharynx, or larynx. In other embodiments, the head and neck cancer is a salivary gland tumor, lymphoma, or sarcoma. The patient may require radiation treatment at one, or a plurality of oral sites. Such radiation therapy targeting head and neck tumors, has the effect, conventionally, of inducing OM. In addition to radiation therapy, the patient may also receive a chemotherapy regimen, such as with a chemotherapy agent or combination of agents that conventionally induces mucositis. The chemotherapy may comprise, for example, cisplatin, 5-fluorouracil, carboplatin, and/or paclitaxel.

In accordance with such embodiments, the patient is more able to complete the planned course of therapy, without disruption due to the pain and discomfort of mucositis or due to the patient's nutritional status. The planned course of radiation therapy may be, for example, from 3 to 10 weeks in duration, such as about five to about seven weeks in duration. Treatment is generally scheduled for a plurality of times per week, such as about 5 to about 20 times per week. For example, in certain embodiments, the patient receives radiation therapy once or twice per day, at least 5 days per week for the duration of radiation therapy (e.g., for about five to about seven weeks).

The course of radiation therapy involves a cumulative dose of radiation that, conventionally, tends to induce ulcerative or severe OM. For example, in various embodiments the radiation therapy involves a cumulative dose of at least about 30 Gy to at least one oral site. In some embodiments, the radiation therapy involves a cumulative dose of at least about 40 Gy and in yet other embodiments the cumulative dose is at least about 50 Gy or at least about 70 Gy to at least one oral site. A single daily fraction may be from about 1.5 Gy to about 2.5 Gy to at least one oral site. In certain embodiments, the radiation therapy (e.g., the cumulative dose and/or single daily fraction) is directed to two or more oral sites.

The regimen of SCV-07 comprises administration of an SCV-07 dose prior to (approximately) each radiation dose, or (approximately) on each day of radiation treatment. As disclosed herein, the dose of SCV-07 is above the threshold dose effective for preventing or delaying the onset of oral mucositis. For example, the SCV-07 is generally administered at doses of more than at least about 0.02 mg/kg, such as from about 0.1 mg/kg to about 2 mg/kg. In certain embodiments, the dose of SCV-07 is from about 1 mg to about 100 mg.

In other embodiments, the patient is receiving chemotherapy for cancer or malignancy, such as but not limited to head and neck cancer, breast cancer, lung cancer, ovarian cancer, colorectal cancer, or a hematologic malignancy (e.g., non-Hodgkin lymphoma). The hematologic malignancy may involve treatment with a myelotoxic regimen that conventionally induces OM. In some embodiments, the patient is undergoing conditioning therapy (e.g., chemotherapy and/or radiation therapy) for bone marrow transplantation. The cancer patient may be undergoing therapy that comprises any stomatotoxic chemotherapy, such as 5-fluorouracil, methotrexate, and/or cytarabine. Other exemplary agents include one or more of cisplatin, carboplatin, 5-fluorouracil, docetaxel, doxorubicin, etoposide, and/or paclitaxel. In other embodiments, the patient is undergoing radiation therapy.

In accordance with the various embodiments, the invention prevents or delays the onset of OM. In certain embodiments, the invention delays the onset of ulcerative or severe OM. The invention delays the onset of OM defined as WHO scale 1 to 4, or ulcerative OM defined as WHO scale 2 to 4, or severe OM, defined as WHO scale 3 and 4. In some embodiments, the method prevents or delays the need for a gastrostomy feeding tube, thereby helping the patient maintain a satisfactory nutritional state over the course of radiation therapy and/or chemotherapy. In still other embodiments, the method reduces the need for administration of opioids over the course of therapy.

### DESCRIPTION OF THE FIGURES

FIG. 1 illustrates the delay to onset of severe OM (WHO score >2) as a function of cumulative radiation dose in a cohort of patients receiving chemoradiation for head and neck cancer. Patients receiving a regimen of 0.10 mg/kg SCV-07 showed a delay to onset of OM (Full Analysis Set, “FAS”).

FIG. 2 illustrates the delay to onset of severe OM (WHO score >2) as a function of cumulative radiation dose as in FIG. 1. FIG. 2 illustrates the results for patients that completed radiation therapy. Patients receiving a regimen of 0.10 mg/kg SCV-07 showed a delay to onset of OM.

FIG. 3 illustrates the delay to onset of ulcerative OM (WHO score >1) as a function of cumulative radiation dose. Patients receiving a regimen of 0.10 mg/kg of SCV-07 showed a delay to onset of OM (Completer Population).

### DETAILED DESCRIPTION OF THE INVENTION

The methods of the present invention provide for preventing or delaying the onset of oral mucositis. Oral mucositis (OM), or stomatitis, is a common and debilitating complication of cancer chemotherapy and radiation therapy. OM is an inflammatory response of the oral mucosa and intraoral soft tissue structures in the oral cavity that occurs in response to the administration of radiation therapeutics and chemotherapeutics, as well as other cytotoxic therapies. It typically affects the inner surfaces of the cheeks and lips, the floor of the mouth, the lateral surfaces of the tongue and the bottom surfaces of the tongue and the soft palate. Lesions can also occur on the hard palate and upper surface of the tongue. Specifically, OM results from the systemic effects of stomatotoxic chemotherapy agents and from the local effects of radiation directed to the oral mucosa or the oral cavity. Mucositis may also limit the patient's ability to tolerate the full regimen of chemotherapy or radiotherapy, thereby impacting the effectiveness of the treatment. Further, patients with damaged oral mucosa and reduced immunity resulting from chemotherapy and radiotherapy are also prone to opportunistic infections in the mouth. It is therefore critical that OM be prevented or reduced as much as possible.

The present invention provides methods for preventing or delaying the onset of oral mucositis in a patient receiving radiation therapy and/or chemotherapy. The method comprises administering to the patient an effective regimen of γ-D-glutamyl-L-tryptophan (SCV-07) over the course of therapy. The regimen, which includes scheduled doses of SCV-07 with respect to radiation exposure or chemotherapy administration, is effective for preventing or delaying the onset of OM, including the onset of ulcerative or severe OM. In accordance with the invention, the patient is more able to complete the planned course of therapy, by maintaining a sufficient nutritional state, and by avoiding the significant pain and discomfort associated with OM.

The methods of the present invention contemplate the use of SCV-07 for the prevention or delay of the onset of conditions and/or symptoms related to mucositis. Conditions related to mucositis vary from pain and discomfort to an inability to tolerate food or even fluids. In some embodiments, conditions related to OM can include erythema (reddening due to inflammation), swelling (edema), ulcerations, thickening of the keratin layer of the mucosa or skin (hyperkeratosis), a false membrane consisting of exudate and fibrin covering an ulceration (pseudomembranous mucosa), superficial infection caused by a yeast-like fungus of the genus Candida (candidiasis), swollen lymph nodes (lymphadenopathy), herpetic infections, deep fungal infections, bacterial infections, malnutrition (due to pain during eating), dehydration (due to pain during swallowing), bleeding (which can result in thrombocytopenia), the number and/or frequency of hospital and/or clinic visits due to OM, the need for breaks and/or interruptions in chemotherapy and/or radiation therapy and refusal of the radiation therapy and/or chemotherapy treatment regimen.

In certain embodiments, the present invention provides methods for preventing or delaying the onset of a condition selected from erythema, edema, ulcerations, hyperkeratosis, and pseudomembranous mucosa in the oral cavity of a patient. Such methods comprise administering to the patient a regimen of γ-D-glutamyl-L-tryptophan (SCV-07) over the course of radiation therapy and/or chemotherapy wherein the regimen is effective for preventing or delaying the onset of the condition. In some embodiments, the condition prevented or delayed by the methods further includes candidiasis, lymphadenopathy, herpetic infections, deep fungal infections, bacterial infections, malnutrition, dehydration, the number and/or frequency of hospital and/or clinic visits due to OM, the need for breaks and/or interruptions in chemotherapy and/or radiation therapy and refusal of the radiation therapy and/or chemotherapy treatment regimen.

Such conditions associated with oral mucositis affect the oral cavity. In some embodiments, the oral cavity includes the upper and lower lips, right and left cheeks, right and left ventral and lateral tongue, floor of the mouth, soft palate, fauces, buccal mucosa and hard palate.

The progression of OM is categorized in stages, and the methods of the delay or onset of OM in accordance with various embodiments can be determined by reference to these various stages of OM. In some embodiments the methods prevent or delay the onset and/or progression of early signs of mucositis, such as but not limited to mild erythema and edema (such as edema of the buccal mucosa and/or tongue). In other embodiments, the methods of the present invention prevent or delay the onset and/or progression of redness (erythema) and/or inflammation. In yet other embodiments, the methods of the present invention prevent or delay the onset and/or progression of ulcerations (lesions; mouth sores). Such ulcerations can range in size from a few millimeters to a few centimeters long and can occur 7 to 14 days after the radiation therapy or chemotherapeutic is administered. In some cases the ulcerations can up take 7 days or more to heal after the radiation therapy or chemotherapeutic is discontinued. In still other embodiments, the methods of the present invention prevent or delay the onset and/or progression of secondary infections, such as candidiasis, herpetic infections, fungal infections and/or bacterial infections,

There are several scales and methods of categorizing OM, and these scales may be used for determining the time of onset and severity of OM in accordance with the invention. In some embodiments, the categorization is based on the WHO OM Scale, which is a validated instrument for measuring OM. The methods of the present invention provide for preventing or delaying the onset and/or progression of OM categorized as any of WHO grades 1 to 4. Grade 0 represents no OM. Grade 1 indicates erythema and soreness, but no ulcers. Grade 2 indicates the presence of ulcers, but the patient is able to eat a solid diet. Grade 3 indicates the presence of ulcers, extensive erythema and the patient requires a liquid diet. Grade 4 indicates the presence of ulcers, and the patient is not able to tolerate a solid or liquid diet, requiring IV or tube feeding.

Another scale that can be used in categorizing OM includes the RTOG assessment scale. The methods of the present invention provide for preventing or delaying the onset and/or progression of OM categorized as any of RTOG scores 1 to 4. A score of 0 corresponds to no oral mucositis. A score of 1 corresponds to erythema of the mucosa. A score of 2 corresponds to a patchy reaction (<1.5 cm, noncontiguous). A score of 3 corresponds to confluent reaction (>1.5 cm, contiguous). A scale of 4 corresponds to necrosis or deep ulceration present, with or without bleeding.

Yet another scale that can be utilized in categorizing OM is the Western Consortium for Cancer Nursing Research (WCCNR) scale. The methods of the present invention provide for preventing or delaying the onset and/or progression of OM categorized as any of WCCNR scores 1 to 3. A score of 0 corresponds to no lesions, oral cavity pink in color and no bleeding. A score of 1 corresponds to 1-4 lesions, oral cavity slight red in color and no apparent bleeding. A score of 2 corresponds to greater than 4 lesions, oral cavity moderate red in color and spontaneous bleeding. A score of 3 corresponds to coalescing lesions, oral cavity very red in color and spontaneous bleeding.

An additional scale that is useful in categorizing OM is the Oral Mucositis Assessment Scale (OMAS), which allows for assessment of oral mucositis that is based on the presence and size of lesions (ulcerations) or pseudomembranes. The methods of the present invention provide for preventing or delaying the onset and/or progression of OM categorized as any of OMAS scores 1 to 3. A score of 0 corresponds to no lesions. A score of 1 corresponds to lesions less than 1 cm². A score of 2 corresponds to lesions that are about 1 cm² to 3 cm². A score of 3 corresponds to lesions that are larger than 3 cm². The OMAS scale also has a separate scoring system for erythema that ranges from 0 to 2. The methods of the present invention provide for preventing or delaying the onset and/or progression of OM categorized as having an OMAS erythema score of 1 or 2. A score of 0 corresponds to no erythema. A score of 1 corresponds to erythema that is not severe. A score of 2 corresponds to erythema that is severe. Erythema is examined for all the tissues in the oral cavity, including the upper and lower lips, right and left cheeks, right and left ventral and lateral tongue, floor of the mouth, soft palate, fauces, and hard palate.

For references related to scales for measuring OM, see, e.g., Sonis S T, Eilers J P, Epstein J B, et al. Validation of a new scoring system for the assessment of clinical trial research of oral mucositis induced by radiation or chemotherapy. Mucositis Study Group. Cancer. 85:2103-2113 (1999); World Health Organization. Handbook for reporting results of cancer treatment. Geneva, Switzerland: World Health Organization, 15-22 (1979); WCCNR: Assessing stomatitis: refinement of the Western Consortium for Cancer Nursing Research (WCCNR) stomatitis staging system. Can Oncol Nurs J., 8:160-165 (1998); Trotti A, Byhardt R, Stetz J, et al. Common toxicity criteria: version 2.0. An improved reference for grading the acute effects of cancer treatment: impact on radiotherapy. Int J Radiat Oncol Biol Phys., 47:13-47 (2000); National Cancer Institute Common Toxicity Criteria. Version 2.0, Jun. 1, 1999; and Sonis S T, Oster G, Fuchs F, et al. Oral mucositis and the clinical and economic outcomes of hematopoietic stem-cell transplantation. J Clin Oncol. 19:2201-2205 (2001); all of which are incorporated by reference herein in their entirety.

The molecular mechanisms underlying progression of oral mucositis occur in roughly five stages: 1) initiation, 2) up-regulation and message generation, 3) amplification and signaling, 4) ulceration, and 5) healing. In various embodiments, the methods of the present invention may prevent or delay the onset and/or progression of any stage of oral mucositis.

The present invention further provides methods for modulating one or more biological pathways involved in the onset and/or progression of oral mucositis in a patient by administering to the patient a regimen of γ-D-glutamyl-L-tryptophan (SCV-07) wherein the regimen is effective for modulating a biological pathway involved in the onset and/or progression of oral mucositis. In some embodiments, the biological pathway involved in the onset and/or progression of oral mucositis is selected from nitrogen metabolism, Toll-like receptor signaling, NF-κB signaling, B-cell receptor signaling, P13K/AKT signaling, the cell cycle G2/M DNA damage checkpoint receptor, p38 MAPK signaling, Wnt/B-catenin signaling, glutamate receptor signaling, Integrin signaling, VEGF signaling, IL-6 signaling, Death receptor signaling, SAPK/JNK signaling and ceramide pathway.

The present invention also provides methods for modulating a molecular target involved in the onset and/or progression of oral mucositis in a patient by administering to the patient a regimen of γ-D-glutamyl-L-tryptophan (SCV-07) wherein the regimen is effective for modulating a molecular target involved in the onset and/or progression of oral mucositis. In some embodiments, the molecular target is selected from nuclear factor-kappa B (NF-κB), NRF2, SP1-related retinoblastoma control protein, p53, interleukin (IL)1-β, tumor necrosis factor (TNF) and interleukin-6 (IL-6).

SCV-07 (γ-D-glutamyl-L-tryptophan) is a small molecule which is thought to stimulate the immune system through, for example, inhibition of STAT3 signaling and the resulting effects on T-helper 1 cells. γ-D-glutamyl-L-tryptophan and its immune stimulating properties are described in U.S. Pat. No. 5,916,878, which is hereby incorporated by reference in its entirety. The utility of SCV-07 for the treatment or prevention of tissue deterioration, injury or damage (e.g., by radiation treatment) is disclosed in WO 2008/100458, which is hereby incorporated by reference in its entirety.

In accordance with the invention, the patient receives treatment (e.g., chemotherapy and/or radiation therapy) for a disorder, such as cancer. Where the patient is a cancer patient, the cancer may be of any stage (e.g., Stage III or Stage IV). In some embodiments, the patient receives radiation therapy for head and neck cancer. For example, the cancer may be a squamous cell carcinoma of the oral cavity, oropharynx, hypopharynx or larynx. In other embodiments, the head and neck cancer is a salivary gland tumor, lymphoma or sarcoma. The patient may require radiation surgery at one, or a plurality of oral sites. Radiation therapy targeting such tumors, has the effect of (conventionally) inducing OM.

In addition to or as an alternative to such radiation therapy, in some embodiments the patient may receive a chemotherapy regimen, such as a chemotherapy that conventionally induces mucositis (stomatotoxin). The chemotherapy may be, for example but is not limited to, paclitaxel, doxorubicin, mithramycin, docetaxel, platinum-based chemotherapeutics (including but not limited to cisplatin and carboplatin), mitomycin, methotrexate, fluorouracil, 5-fluorouracil (5-FU), vinorelbine, topotecan, irinotecan, bleomycin, bleomycin hydrorxyurea, mitomycin, actinomycin, topoisomerase I and II inhibitors, anthracylines, epirubicin, idarubicin, mitoxantrone, valrubicin, etoposide, teniposide, rubitecan, and derivatives thereof. The chemotherapy may include a taxane and/or an antimetabolite and/or derivatives thereof.

In other embodiments, the patient is receiving chemotherapy for cancer or malignancy, such as carcinoma, sarcoma, blastoma, lymphoma, leukemia, and germ cell tumors. In some embodiments, the chemotherapy may be administered in the absence of radiation therapy. In yet other embodiments, the cancer or malignancy can include but is not limited to head and neck cancer, breast cancer, ovarian cancer, lung cancer, colorectal cancer, skin cancer, oral cancer, glioblastoma, laryngeal cancer, esophageal cancer, endothelial cancer, endometrial cancer, urogenital cancer, rectal cancer, prostate cancer, kidney cancer, melanoma, renal cancer, and papilloma virus-induced cancer, or other type of cancer, such as a hematologic malignancy (e.g., non-Hodgkin lymphoma). The hematologic malignancy may involve treatment with a myelotoxic regimen that conventionally induces OM. In some embodiments, the patient is undergoing conditioning therapy (e.g., chemotherapy and/or radiation therapy) for bone marrow transplantation. The cancer patient may be undergoing therapy that comprises a stomatotoxic chemotherapy, such as 5-fluorouracil, methotrexate, and cytarabine. Other agents include cisplatin, carboplatin, 5-fluorouracil, docetaxel, doxorubicin, etoposide, and/or paclitaxel.

Patients undergoing conventional radiation therapy to the head and neck typically experience erythema and mouth soreness within about two weeks of beginning therapy and often develop more severe damage to the oral epithelium within the following two weeks. When both chemotherapy and radiotherapy are administered, the incidence and severity of oral mucositis can be exacerbated.

In accordance with the present invention, in some embodiments the patient receives radiation therapy for head and neck cancer, along with a regimen of SCV-07. The radiation therapy may include External beam therapy (EBT) or Intensity-modulated radiation therapy (IMRT). EBT delivers a beam of high-energy x-rays to the location of the tumor. The beam is generated outside the patient (usually by a linear accelerator) and is targeted at the tumor site. These x-rays can destroy the cancer cells and careful treatment planning allows the surrounding normal tissues to be spared. No radioactive sources are placed inside the patient's body. IMRT is an advanced mode of high-precision radiotherapy that utilizes computer-controlled x-ray accelerators to deliver precise radiation doses to a malignant tumor or specific areas within the tumor. The radiation dose is designed to conform to the three-dimensional (3-D) shape of the tumor by modulating, or controlling, the intensity of the radiation beam to focus a higher radiation dose to the tumor while minimizing radiation exposure to healthy cells. Brachytherapy can also be employed, which uses sealed radioactive sources implanted into the treatment area which can be either temporary or permanent.

Typically, radiation treatments are given once or twice a day, about five days a week for five to seven weeks. In certain embodiments of the invention, the course of radiation therapy is from 3 to 10 weeks in duration. The course of radiation therapy may be about five to about seven weeks in duration. The course of radiation therapy may involve radiation treatment from about 5 to about 20 times per week. The course of radiation therapy may involve radiation treatment from about 7 to about 15 times per week. The patient may receive radiation therapy once or twice per day, at least 5 days per week, for from five to about seven weeks.

The course of radiation therapy may involve a cumulative dose of at least about 30 Gy to at least one oral site. In some embodiments, the course of radiation therapy is a cumulative dose of at least about 40 Gy, about 50 Gy, about 60 Gy, or about 70 Gy to at least one oral site. Thus, the course of radiation therapy may involve a cumulative dose of from about 50 to about 75 Gy to at least one oral site. In such embodiments, a single daily fraction is from about 1.5 Gy to about 2.5 Gy to at least one oral site. Of course the daily fraction and cumulative dose may be directed to two or more oral sites, including 3 or 4 oral sites.

In some embodiments, the patient receives both chemotherapy and radiation therapy. The chemotherapy may involve a stomatotoxic compound, including but not limited to a taxane, antimetabolite, and/or antibiotic. In various embodiments, the chemotherapy includes administration of cisplatin, fluorouracil, carboplatin, and/or paclitaxel. Where the chemotherapy comprises cisplatin, the patient may receive the cisplatin at about 80 to about 100 mg/m² from two to about four times per month. For example, the patient may receive about 80 to about 100 mg/m² of cisplatin approximately tri-weekly (once every three weeks). Where cisplatin is administered in approximately weekly doses, the dose may be in the range of about 30 to 40 mg/m².

In other embodiments, chemotherapy may be given during the course of radiation therapy, since such may be more effective than if given before a course of radiation therapy. In particular, where the cancer is advanced (advanced stage III or stage IV) the radiation treatment schedules sometimes include chemotherapy. Drugs most commonly given in conjunction with radiation therapy are cisplatin (Platinol) and Cetuximab (Erbitux). Occasionally, other drugs may include fluorouracil (5-FU, Adrucil), carboplatin (Paraplatin), and paclitaxel (Taxol). The chemotherapy may be given in a variety of ways, including a low daily dose, a moderately low weekly dose, or a relatively higher dose every three to four weeks.

The regimen of SCV-07 may involve doses of SCV-07 on each day of radiation treatment, or (approximately) before each radiation treatment, and/or during a course of chemotherapy administration. As disclosed herein, the dose of SCV-07 is above the threshold dose effective for preventing or delaying the onset of oral mucositis. The dose of SCV-07 is generally greater than 0.02 mg/kg. The dose of SCV-07 may be at least about 0.1 mg/kg, or at least about 0.3 mg/kg, or at least about 1.0 mg/kg. In other embodiments the dosage of SCV-07 may be in the range of more than about 0.02 mg/kg to about 10 mg/kg. In still other embodiments, the dose of SCV-07 may be in the range of about 0.1 mg/kg to about 2 mg/kg. In yet other embodiments the dose of SCV-07 may be in the range of more than about 0.02 mg/kg to about 2 mg/kg. In still other embodiments the dose of SCV-07 may be in the range of about 0.1 mg/kg to about 1 mg/kg. In various other embodiments, the dose of SCV-07 is from about 1 mg to about 100 mg, or from about 10 mg to about 100 mg, or about 25 mg to about 100 mg. For example, exemplary SCV-07 doses, which may be independently selected over the course of therapy, are about 10 mg, about 25 mg, about 50 mg, about 75 mg, and about 100 mg. In certain embodiments, a dose of SCV-07 is also administered on days in which no radiation and/or chemotherapy is administered, to maintain the relatively constant level of active agent in the patient's system.

In certain embodiments, the SCV-07 dose is split between a plurality of subdoses, with each subdose administered on the day of treatment.

The SCV-07 may be administered by any suitable route, including orally, transdermally, nasally, sublingually, by infusion (e.g., intravenous), by intramuscular injection, or by subcutaneous injection. In certain embodiments, the SCV-07 is administered parenterally, such as by subcutaneous injection.

The SCV-07 active agent may be in the form of a pharmaceutically acceptable hydrate, salt or solvate. The pharmaceutically acceptable salt may be a salt with any non-toxic, organic or inorganic acid. Exemplary organic acids that form suitable salts include hydrochloric, hydrobromic, sulphuric and phosphoric acid, and acid metal salts such as sodium monohydrogen orthophosphate and potassium hydrogen sulfate. Exemplary organic acids that form suitable salts include the mono-, di-, and tricarboxylic acids. For example, the organic acid may be acetic, glycolic, lactic, pyruvic, malonic, succinic, glutaric, fumaric, malic, tartaric, citric, ascorbic, benzoic, hydroxygenzoic, phenylacetic, cinnamic, salicylic, 2-phenoxybenzoic and sulfonic acids. Salts of the carboxylic groups include the non-toxic carboxylic acid salts formed with any suitable inorganic or organic bases. Such salts include alkali metals (e.g., sodium and potassium), alkaline earth metals (e.g., calcium and magnesium), light metals of Group IIIA (e.g., aluminum), and organic primary, secondary, and tertiary amines (e.g., trialkyl-amines, including triethylamine, procaine, dibenzylamine, 1-ethenamine, N,N-dibenzyl-ethylenediamine, dihydroabiethylamine, (N-(lower)alkyl-piperidine, and any other suitable amine) In some embodiments, SCV-07 is administered as a sodium salt.

In accordance with the invention, the patient does not develop oral mucositis during the course of therapy. For example, where the patient receives radiation therapy for head and neck cancer, the patient may not develop ulcerative or severe OM during at least the first 3 weeks of radiation therapy, or during the first 5 weeks of radiation therapy, or during the full course of radiation therapy (e.g., a seven-week course). Thus, the patient may not develop an oral mucositis score of WHO scale 2 to 4, or WHO scale 3 or 4 (or comparable score from another grading scale), during the course of radiation therapy.

In some embodiments, the patient does not use a gastrostomy feeding tube during at least the first 4 weeks of radiation therapy, or during at least the first 5 weeks of radiation therapy, or does not use a gastrostomy feeding tube during the course of radiation therapy.

In some embodiments, the patient is not administered an opioid during at least the first 3 weeks of radiation therapy, or during at least the first 5 weeks of radiation therapy, or is not administered an opioid during the course of radiation therapy. Opioids include drugs such as buprenorphine, codeine, fentanyl and morphine.

In various embodiments, the planned course of radiation therapy and/or chemotherapy is not disrupted due to oral mucositis. In other embodiments, the methods of the present invention reduce the number and/or frequency of hospital and/or clinic visits due to OM. In still other embodiments, the methods reduce the need for breaks and/or interruptions in chemotherapy and/or radiation therapy. In yet other embodiments, the methods of the present invention reduce refusal of the radiation therapy and/or chemotherapy treatment regimen.

In certain exemplary embodiments, a patient having head and neck cancer, and receiving chemoradiation treatment for the head and neck cancer, receives a dose of at least 0.1 mg/kg SCV-07 on at least each day of radiation therapy.

In particular exemplary embodiments, a patient having head and neck cancer, and receiving chemoradiation treatment (comprising a cumulative dose of at least about 40 Gy or at least 50 Gy and a course of cisplatin) for the head and neck cancer, receives a dose of at least 0.1 mg/kg SCV-07 on at least each day of therapy.

### EXAMPLES

### Example 1

### Safety and Efficacy of γ-D-Glutamyl-L-Tryptophan as an Intervention for Oral Mucositis in Patients Receiving Chemoradiation for the Treatment of Cancers of the Head and Neck

OM is a painful, debilitating, and costly toxicity of different chemoradiotherapy (CT/RT) regimens used to treat head and neck cancer. This trial is a phase 2, randomized, double-blind, dose-ranging, placebo-controlled three-arm study in patients receiving CT/RT for the treatment of head and neck cancer, to assess the safety and tolerability of γ-D-glutamyl-L-tryptophan (SCV-07) as well as its efficacy in delaying the onset of severe OM as assessed by the WHO Oral Mucositis Scale.

The WHO OM Scale is the standard, validated instrument used in clinical trials to measure OM: Grade 0=none; Grade 1=erythema and soreness, no ulcers; Grade 2=ulcers, able to eat a solid diet; Grade 3=ulcers; requires a liquid diet; Grade 4=ulcers, not able to tolerate a solid or liquid diet, requires IV or tube feeding.

59 eligible patients with recently diagnosed, treatment naïve, pathologically-confirmed, non-metastatic squamous cell carcinoma of the oral cavity, oropharynx, hypopharynx or larynx were enrolled and treated with a concomitant regimen of CT/RT consisting of a continuous course of external beam irradiation (IMRT eligible) with a minimum cumulative dose of 50 Gray (Gy) and cisplatin monotherapy administered using standard weekly or tri-weekly dosing regimens (80-100 mg/m² administered on Days 0, 21 and 42) or weekly (30-40 mg/m²). At least two ‘at risk’ intraoral sites were included in the radiation fields and received a cumulative RT dose of at least 50 Gy. Subjects were equally randomized to receive either placebo or one of two doses of γ-D-glutamyl-L-tryptophan (0.02 or 0.1 mg/kg) on each radiation day for up to 7 weeks. The patient demographics are shown in Table 1.

Patients receiving the study's higher dose (0.1 mg/kg) of SCV-07 showed a trend towards delay to severe OM (WHO scale, grades 3 and 4), the study's primary endpoint. Patients in the low dose treatment arm (0.02 mg/kg) appeared to do worse than placebo, showing that treatment effect is sensitive to dose. Additionally, SCV-07 was safe and well tolerated with no drug-related serious adverse events reported, indicating that there is potential to administer higher doses of SCV-07.

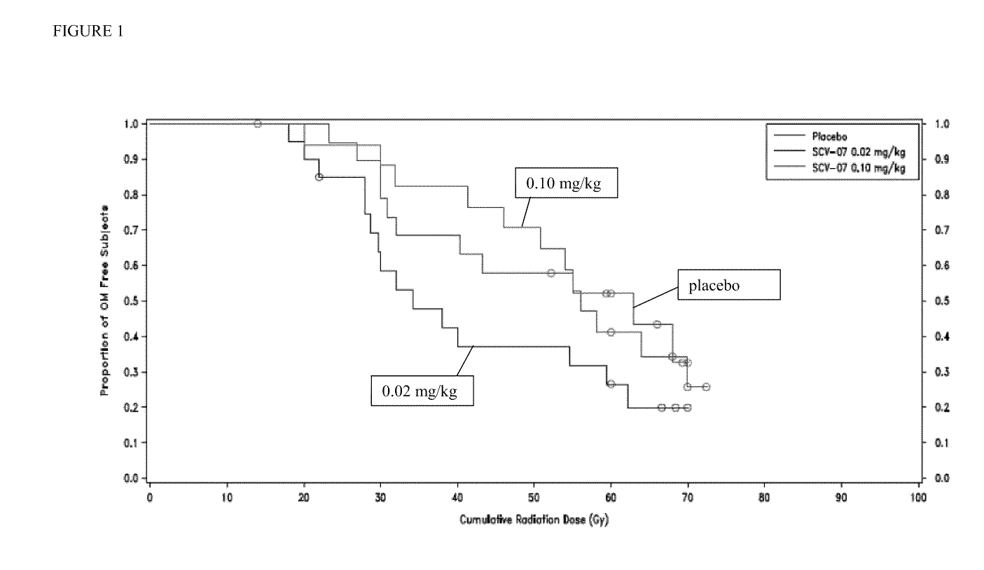

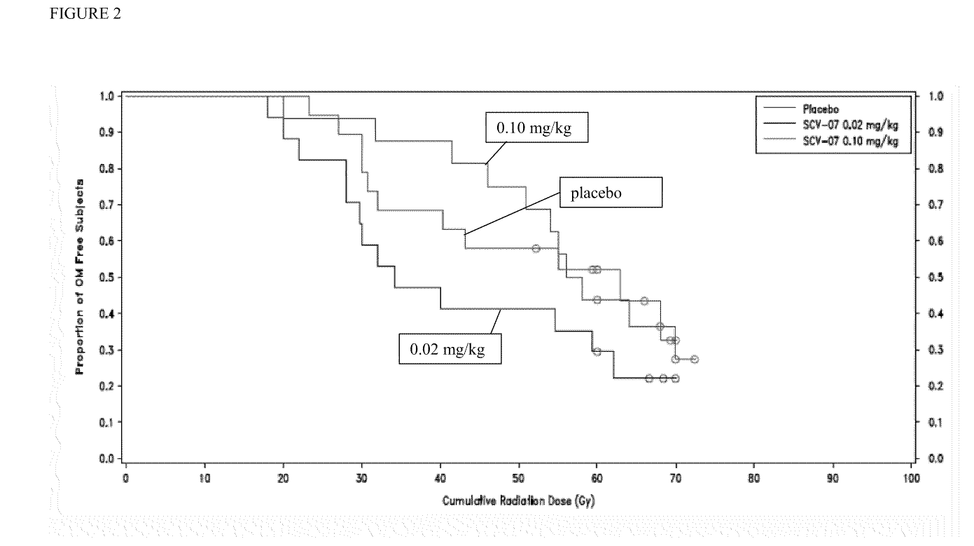

Tables 2-7 summarize the statistics for the Full Analysis Set “FAS” (57 subjects), and the completer population (52 subjects), with respect to delay of onset of severe oral mucositis. Also see FIGS. 1 and 2.

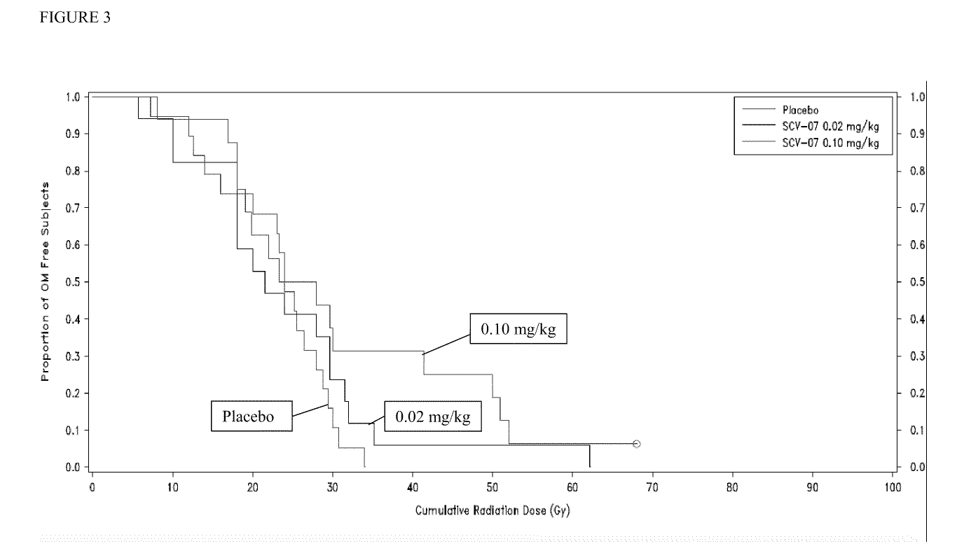

Additional data analysis showed a more pronounced clinical benefit for patients in the high dose treatment arm when evaluating the delay to onset of ulcerative OM (WHO scale, grades 2 to 4), an expanded measure of OM (see FIG. 3 and Tables 8-10). In this analysis, the low dose treatment arm appeared similar or slightly better than placebo. Ulceration is the major cause of the morbidity associated with OM.

Table 11 below shows that patients receiving SCV-07 were less likely to require placement of a gastrostomy feeding tube. Patients receiving the high dose of SCV-07 used the gastostomy tube fewer days than patients receiving placebo and patients in the low treatment arm.

TABLE 11

| | | |
|---|---|---|
| | SCV-07 low dose | SCV-07 high dose |
| Placebo (n = 6/20) | (n = 4/20) | (n = 3/20) |

Percent of Patients with G-Tube Placement

| | | |
|---|---|---|
| 30 | 20 | 18 |

Number of Days that Patients used the G-Tube

| | | |
|---|---|---|
| 51 | 57 | 44 |

In conclusion, γ-D-glutamyl-L-tryptophan was safe and well tolerated. Subjects given the higher dose of γ-D-glutamyl-L-tryptophan showed a trend towards delay in onset of severe OM (WHO grade >2) and any ulcerative OM (WHO ≧2), fewer unplanned office and emergency room visits, and less reliance on gastrostomy tube placement and use.

### Example 2

### Development of γ-D-Glutamyl-L-Tryptophan (SVC-07) for the Treatment of Oral Mucositis (OM)

SCV-07 efficacy, dosing and scheduling parameters were assessed in hamster models of acute, fractionated and concomitant chemoradiation (CRT). Xenograft studies using human head and neck cancer (HNC) lines confirmed that SCV-07 did not impact CRT anti-tumor response. A multicenter, prospective, blinded, randomized trial evaluated SCV-07's ability to alter OM in CRT-treated HNC patients (n=57). Cytokine and gene microarray analyses were performed for samples obtained before and on the last radiation day.

Subcutaneous SCV-07 at a dose of 0.1 mg/kg (n=17; HD), but not 0.02 mg/kg (n=19; LD), delayed severe OM vs. placebo (n=20)[18% vs. 32% at ≦40Gy and 29% vs. 42% at 50 Gy] and the onset of ulcerative OM (UOM). Cox regression analysis of time to UOM initial occurrence demonstrated a 52% decrease in the HD SCV-07 cohort vs. placebo. HD SCV-07 resulted in fewer G-tubes placed, unplanned or emergent visits, and treatment breaks. Elevated macrophage-associated cytokines confirmed the biological activity in SCV-07-treated patients. Increases in the macrophage cytokines MIF and MIP-1beta were observed. Decreases in the proinflammatory cytokines IL-1alpha, MIP-3 alpha, TNF-alpha, IL-5 and IL-15 were observed. Increases were also observed in VCAM, ICAM and IL-12. Furthermore, persons who clinically responded to SCV-07 showed a decrease in VEGF and increases in MIF and IL-21, a Th1-associated cytokine from CD4⁺ cells. Pro-inflammatory cytokine levels were reduced. Gene expression differences were noted between SCV-07 and placebo patients. A unique cluster of genes was identified that discriminated SCV-07 responders from non-responders.

Accordingly, SCV-07 can be an effective, therapy to attenuate CRT induced OM in HNC patients.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

TABLE 1
Demographics and Baseline Characteristics - Completer Population

| | | SCV-07 | | |
|---|---|---|---|---|
| | Placebo | 0.02 mg/kg | 0.10 mg/kg | Total |
| | (N = 19) | (N = 17) | (N = 16) | (N = 52) |
| Age at | | | | |
| consent (yrs) | | | | |
| N | 19 | 17 | 16 | 52 |
| Mean (SD) | 56.3 (10.60) | 58.2 (10.04) | 54.4 (9.00) | 56.3 (9.87) |
| Median | 54.0 | 57.0 | 52.5 | 54.0 |
| Min, Max | 42.0, 84.0 | 43.0, 77.0 | 43.0, 76.0 | 42.0, 84.0 |
| Gender | | | | |
| Male | 13 (68%) | 14 (82%) | 9 (56%) | 36 (69%) |
| Female | 6 (32%) | 3 (18%) | 7 (44%0 | 16 (31%) |
| Race | | | | |
| White | 18 (95%) | 17 (100%) | 13 (81%) | 48 (92%) |
| Back or | 1 (5%) | 0 | 3 (19%) | 4 (8%) |
| African | | | | |
| American | | | | |
| Asian | 0 | 0 | 0 | 0 |
| American | 0 | 0 | 0 | 0 |
| Indian or | | | | |
| Alaska Native | | | | |
| Native | 0 | 0 | 0 | 0 |
| Hawaiian or | | | | |
| Other Pacific | | | | |
| Islander | | | | |
| Other | 0 | 0 | 0 | 0 |
| Ethnicity | | | | |
| Hispanic | 1 (5%) | 4 (24%) | 1 (6%) | 6 (12%) |
| Not Hispanic | 18 (95%) | 13 (76%) | 15 (94%) | 46 (88%) |
| Months since | | | | |
| curative | | | | |
| surgery [1] | | | | |
| N | 6 | 6 | 4 | 16 |
| Mean (SD) | 1.3 (0.34) | 1.4 (0.12 ) | 2.4 (2.58) | 1.6 (1.25) |
| Median | 1.3 | 1.3 | 1.4 | 1.4 |
| Min, Max | 1.0, 1.8 | 1.2, 1.6 | 0.5, 6.2 | 0.5, 6.2 |

TABLE 2
Analyses of the Primary and Second Efficacy Endpoint: Severe
OM (Who > 2) - FAS population

| Cum. Percent of | | SCV-07 | | |
|---|---|---|---|---|
| Subjects with | Placebo | 0.02 mg/kg | 0.10 mg/kg | Total |
| Severe OM at: | (N = 20) | (N = 20) | (N = 17) | (N = 57) |
| >30 Gy | 11% (3%, 36%) | 36% (19%, 61%) | 6% (1%, 35%) | 18% (10%, 31%) |
| 30-<35 Gy | 32% (16%, 57%) | 52% (32%, 75%) | 18% (6%, 45%) | 34% (24%, 49%) |
| 35-<40 Gy | 32% (16%, 57%) | 58% (37%, 79%) | 18% (6%, 45%) | 36% (25%, 50%) |
| 40-<45 Gy | 42% (24%, 67%) | 63% (42%, 83%) | 24% (10%, 51%) | 44% (32%, 58%) |
| 45-<50 Gy | 42% (24%, 67%) | 63% (42%, 83%) | 29% (13%, 57%) | 45% (33%, 59%) |
| 50-<55 Gy | 42% (24%, 67%) | 68% (47%, 87%) | 41% (22%, 67%) | 51% (39%, 65%) |
| 55-<60 Gy | 48% (28%, 72%) | 73% (53%, 90%) | 59% (37%, 81%) | 60% (48%, 73%) |
| 60-<65 Gy | 57% (35%, 81%) | 80% (59%, 94%) | 66% (44%, 87%) | 68% (55%, 80%) |
| 65-<70 Gy | 67% (42%, 90%) | 80% (59%, 94%) | 74% (51%, 93%) | 75% (61%, 87%) |
| ≧70 Gy | 67% (42%, 90%) | 80% (59%, 94%) | 74% (51%, 93%) | 75% (61%, 87%) |

TABLE 3
Analyses of the Primary and Secondary Efficacy Endpoint: Severe OM (Who > 2) - FAS Population

| | | SCV-07 | | | |
|---|---|---|---|---|---|
| | Placebo | 0.02 mg/kg | 0.10 mg/kg | Total | |
| | (N = 20) | (N = 20) | (N = 17) | (N = 57) | p-value |
| Cumulative Dose of Radiation (Gy) Prior to | | | | | p = 0.149 |
| Initial Occurrence of Severe OM (Delay to | | | | | |
| Onset of Severe OM) [1] | | | | | |
| Summary Statistics (K-M Estimate) | | | | | |
| N (Censored) | 20 (9) | 20 (5) | 17 (5) | 57 (19) | |
| Mean (SE) | 52 (4.2) | 41 (3.8) | 54 (3.9) | 50 (2.5) | |
| Median (95% CI) | 63 (32, NA) | 34 (29, 59) | 56 (46, 70) | 55 (40, 63) | |
| 25%-75% | 31-NA | 28-62 | 46-NA | 30-NA | |
| Pairwise Comparisons | | | | | |
| SCV-07 0.02 mg/kg vs placebo | p = 0.103 | | | | |
| SCV-07 0.10 mg/kg vs placebo | p = 0.879 | | | | |

TABLE 4
Analyses of the Primary and Secondary Efficacy Endpoint: Severe OM (Who > 2) - FAS Population

| | | SCV-07 | | | |
|---|---|---|---|---|---|
| | Placebo | 0.02 mg/kg | 0.10 mg/kg | Total | |
| | (N = 20) | (N = 20) | (N = 17) | (N = 57) | p-value |
| Cox Regression Analysis of Time (Days) to | | | | | p = 0.161 |
| Initial Occurrence of Severe OM [2] | | | | | |
| Summary Statistics (K-M Estimate) | | | | | |
| N (Censored) | 20 (9) | 20 (5) | 17 (5) | 57 (19) | |
| Mean (SE) | 37 (3.3) | 28 (2.7) | 37 (2.7) | 35 (1.9) | |
| Median (95% CI) | 41 (23, NA) | 24 (19, 41) | 40 (32, NA) | 36 (27.45) | |
| 25%-7% | 21-NA | 18-42 | 32-NA | 21-NA | |
| Hazard Ratio (95% CI) | | | | | |
| SCV-07 0.02 mg/kg vs placebo | 1.989 (0.908, 4.357) | | | | |
| SCV-07 0.10 mg/kg vs placebo | 1.100 (0.484, 2.500) | | | | |
| Cisplatin Schedule | 1.489 (1.004, 2.209) | | | | |
| Cumulative Dose of RT (Gy) at Initial | | | | | p = 0.123 |
| Onset of Severe OM [3] | | | | | |
| N | 20 | 20 | 17 | 57 | |
| Mean (SD) | 48 (18.3) | 41 (18.0) | 54 (15.6) | 47 (18.0) | |
| Median | 54 | 33 | 56 | 52 | |
| Min, Max | 14, 70 | 18, 70 | 20, 72 | 14, 72 | |

TABLE 5
Analyses of the Primary and Secondary Efficacy Endpoint: Severe
OM (WHO > 2) - Completer Population

| | | SCV-07 | | |
|---|---|---|---|---|
| Cum. Percent of Subjects | Placebo | 0.02 mg/kg | 0.10 mg/kg | Total |
| with Severe OM at: | (N = 19) | (N = 17) | (N = 16) | (N = 52) |
| <30 Gy | 11% (3%, 36%) | 35% (18%, 62%) | 6% (1%, 37%) | 17% (9%, 31%) |
| 30-<35 Gy | 32% (16%, 57%) | 53% (32%, 77%) | 13% (3%, 41%) | 33% (22%, 47%) |
| 35-<40 Gy | 32% (16%, 57%) | 53% (32%, 77%) | 13% (3%, 41%) | 33% (22%, 47%) |
| 40-<45 Gy | 42% (24%, 67%) | 59% (37%, 81%) | 19% (6%, 48%) | 40% (29%, 55%) |
| 45-<50 Gy | 42% (24%, 67%) | 59% (37%, 81%) | 25% (10%, 54%) | 42% (30%, 57%) |
| 50-<55 Gy | 42% (24%, 67%) | 65% (43%, 86%) | 38% (19%, 65%) | 48% (36%, 63%) |
| 55-<60 Gy | 48% (28%, 72%) | 71% (49%, 89%) | 56% (34%, 80%) | 58% (45%, 72%) |
| 60->65 Gy | 57% (35%, 81%) | 78% (56%, 94%) | 64% (41%, 86%) | 66% (53%, 79%) |
| 65-<70 Gy | 67% (42%, 90%) | 78% (56%, 94%) | 73% (48%, 92%) | 74% (59%, 86%) |
| ≧70 Gy | 67% (42%, 90%) | 78% (56%, 94%) | 73% (48%, 92%) | 74% (59%, 86%) |

40% fewer subjects get severe OM by end of week 5 when treated with SCV-07 (0.10 mg/kg)

TABLE 6
Analyses of the Primary and Secondary Efficacy Endpoint: Severe
Om (WHO > 2) - Completer Population

| | | SCV-07 | | | |
|---|---|---|---|---|---|
| | Placebo | 0.02 mg/kg | 0.10 mg/kg | Total | |
| | (N = 19) | (N = 17) | (N = 16) | (N = 52) | p-value |
| Cumulative Dose of Radiation (Gy) Prior to | | | | | p = 0.211 |
| Initial Occurrence of Severe OM (Delay to | | | | | |
| Onset of Severe OM) [1] | | | | | |
| Summary Statistics (K-M Estimate) | | | | | |
| N (Censored) | 19 (8) | 17 (4) | 16 (5) | 52 (17) | |
| Mean (SE) | 52 (4.2) | 42 (4.2) | 56 (3.8) | 50 (2.6) | |
| Median (95% CI) | 63 (32, NA) | 34 (28, 62) | 57 (51, NA) | 55 (41, 64) | |
| 25%-75% | 31-NA | 28-62 | 48-NA | 31-NA | |
| Pairwise Comparisons | | | | | |
| SCV-07 0.02 mg/kg vs placebo | p = 0.161 | | | | |
| SCV-07 0.10 mg/kg vs placebo | p = 0.936 | | | | |

TABLE 7
Analyses of the Primary and Secondary Efficacy Endpoint: Severe
OM (Who > 2) - Completer Population

| | | SCV-07 | | | |
|---|---|---|---|---|---|
| | Placebo | 0.02 mg/kg | 0.10 mg/kg | Total | |
| | (N = 19) | (N = 17) | (N = 16) | (N = 52) | p-value |
| Cox Regression Analysis of Time (Days) to | | | | | p = 0.203 |
| Initial Occurrence of Sever OM [2] | | | | | |
| Summary Statistics (K-M Estimate) | | | | | |
| N (Censored) | 19 (8) | 17 (4) | 16 (5) | 52 (17) | |
| Mean (SE) | 37 (3.3) | 28 (3.0) | 38 (2.5) | 35 (1.9) | |
| Median (95% CI) | 41 (23, NA) | 25 (18, 42) | 43 (33, NA) | 40 (31, 46) | |
| 25%-7% | 21-NA | 18-42 | 33-NA | 21-NA | |
| Hazard Ratio (95% CI) | | | | | |
| SCV-07 0.02 mg/kg vs placebo | 1.884 (0.840, 4.225) | | | | |
| SCV-07 0.10 mg/kg vs placebo | 1.002 (0.433, 2.319) | | | | |
| Cisplatin Schedule | 1.696 (1.087, 2.644) | | | | |
| Cumulative Dose of RT (Gy) at Initial | | | | | p = 0.170 |
| Onset of Severe OM [3] | | | | | |
| N | 19 | 17 | 16 | 52 | |
| Mean (SD) | 50 (16.9) | 43 (18.7) | 55 (14.8) | 49 (17.4) | |
| Median | 55 | 34 | 57 | 55 | |
| Min, Max | 23, 70 | 18, 70 | 20, 72 | 18, 72 | |

TABLE 8
Analyses of Ulcerative OM (WHO > 1) - Completer Population

| | | SCV-07 | | |
|---|---|---|---|---|
| Cum. Percent of Subjects | Placebo | 0.02 mg/kg | 0.10 mg/kg | Total |
| with Ulcerative OM at: | (N = 19) | (N = 17) | (N = 16) | (N = 52) |
| <5 Gy | 0% (0%, 0% | 0% (0%, 0%) | 0% (0%, 0%) | 0% (0%, 0%) |
| 5-<10 Gy | 5% (1%, 32%) | 6% (1%, 35%) | 6% (1%, 37%) | 6% (2%, 17%) |
| 10-<15 Gy | 21% (8%, 47%) | 18% (6%, 45%) | 6% (1%, 37%) | 15% (8%, 28%) |
| 15-<20 Gy | 26% (12%, 52%) | 41% (22%, 67%) | 38% (19%, 65%) | 35% (23%, 49%) |
| 20-<25 Gy | 53% (33%, 76%) | 59% (37%, 81%) | 50% (29%, 75%) | 54% (41%, 68%) |
| 25-<30 Gy | 84% (65%, 96%) | 76% (55%, 93%) | 63% (40%, 85%) | 75% (63%, 86%) |
| 30-<35 Gy | 100% (79%, 100%) | 88% (69%, 98%) | 69% (46%, 89%) | 87% (76%, 94%) |
| 35-<40 Gy | 100% (79%, 100%) | 94% (76%, 100%) | 69% (46%, 89%) | 88% (78%, 95%) |
| 40-<45 Gy | 100% (79%, 100%) | 94% (76%, 100%) | 75% (53%, 92%) | 90% (81%, 96%) |
| 45-<50 Gy | 100% (79%, 100%) | 94% (76%, 100%) | 75% (53%, 92%) | 90% (81%, 96%) |
| 50-<55 Gy | 100% (79%, 100%) | 94% (76%, 100%) | 94% (75%, 100%) | 96% (88%, 99%) |
| 55-<60 Gy | 100% (79%, 100%) | 94% (76%, 100%) | 94% (75%, 100%) | 96% (88%, 99%) |
| 60-<65 Gy | 100% (79%, 100%) | 100% (76%, 100%) | 94% (75%, 100%) | 98% (91%, 100%) |
| 65-<70 Gy | 100% (79%, 100%) | 100% (76%, 100%) | 94% (75%, 100%) | 98% (91%, 100%) |
| ≧70 Gy | 100% (79%, 100%) | 100% (76%, 100%) | 94% (75%, 100%) | 98% (91%, 100%) |

TABLE 9
Analyses of Ulcerative OM (Who > 1) - Completer Population

| | | SCV-07 | | | |
|---|---|---|---|---|---|
| | Placebo | 0.02 mg/kg | 0.10 mg/kg | Total | |
| | (N = 19) | (N = 17) | (N = 16) | (N = 52) | p-value |
| Cumulative Dose of Radiation (Gy) Prior to | | | | | p = 0.085 |
| Initial Occurrence of Ulcerative OM (Delay | | | | | |
| to Onset of Ulcerative OM) [1] | | | | | |
| Summary Statistics (K-M Estimate) | | | | | |
| N (Censored) | 19 (0) | 17 (0) | 16 (1) | 52 (1)) | |
| Mean (SE) | 23 (1.7) | 24 (3.1) | 30 (3.7) | 26 (1.7) | |
| Median (95% CI) | 24 (20, 28) | 22 (18, 30) | 26 (19, 41) | 24 (20, 28) | |
| 25%-75% | 16-29 | 18-30 | 19-46 | 18-30 | |
| Pairwise Comparisons | | | | | |
| SCV-07 0.02 mg/kg vs placebo | p = 0.207 | | | | |
| SCV-07 0.10 mg/kg vs placebo | p = 0.063 | | | | |

TABLE 10
Analyses of Ulcerative OM (Who > 1) - Completer Population

| | | SCV-07 | | | |
|---|---|---|---|---|---|
| | Placebo | 0.02 mg/kg | 0.10 mg/kg | Total | |
| | (N = 19) | (N = 17) | (N = 16) | (N = 52) | p-value |
| Cox Regression Analysis of Time (Days) to | | | | | p = 0.149 |
| Initial Occurrence of Ulcerative OM[2] | | | | | |
| Summary Statistics (K-M Estimate) | | | | | |
| N (Censored) | 19 (0) | 17 (0) | 16 (1) | 52 (1) | |
| Mean (SE) | 14 (1.1) | 16 (2.2) | 20 (2.7) | 17 (1.2) | |
| Median (95% CI) | 15 (13, 17) | 15 (10, 20) | 16 (13, 31) | 15 (14, 17) | |
| 25%-7% | 10-17 | 10-20 | 12-32 | 10-20 | |
| Hazard Ratio (95% CI) | | | | | |
| SCV-07 0.02 mg/kg vs placebo | 0.737 (0.377, 1.444) | | | | |
| SCV-07 0.10 mg/kg vs placebo | 0.475 (0.225, 1.004) | | | | |
| Cisplatin Schedule | 1.494 (1.064, 2.098) | | | | |
| Cumulative Dose of RT (Gy) at Initial | | | | | p = 0.473 |
| Onset of Ulcerative OM [3] | | | | | |
| N | 19 | 17 | 16 | 52 | |
| Mean (SD) | 23 (7.3) | 24 (12.9) | 31 (16.6) | 26 (12.9) | |
| Median | 24 | 22 | 26 | 24 | |
| Min, Max | 7, 34 | 6, 62 | 8, 68 | 6, 68 | |

1. A method for preventing or delaying the onset of oral mucositis in a patient receiving radiation therapy and/or chemotherapy, comprising, administering to the patient a regimen of γ-D-glutamyl-L-tryptophan (SCV-07) over the course of radiation therapy and/or chemotherapy, the regimen being effective for preventing or delaying the onset of oral mucositis.

2. A method for preventing or delaying the onset of a condition or symptom resulting from radiation and/or chemotherapy in the oral cavity of a patient comprising, administering to the patient a regimen of γ-D-glutamyl-L-tryptophan (SCV-07) over the course of radiation therapy and/or chemotherapy, the regimen being effective for preventing or delaying the onset of said condition or symptom, wherein said condition or symptom is selected from erythema, edema, ulcerations, hyperkeratosis, and pseudomembranous mucosa.

3. The method of claim 2, wherein said condition or symptom further includes a condition or symptom selected from candidiasis, lymphadenopathy, herpetic infections, deep fungal infections, bacterial infections, malnutrition, dehydration and refusal or interruption of the radiation therapy or chemotherapeutic treatment regimen.

4. A method for modulating a biological pathway involved in the onset and/or progression of oral mucositis in a patient comprising, administering to a patient receiving radiation therapy and/or chemotherapy a regimen of γ-D-glutamyl-L-tryptophan (SCV-07), the regimen being effective for modulating the biological pathway involved in the onset and/or progression of oral mucositis.

5. The method of claim 4, wherein the biological pathway involved in the onset and/or progression of oral mucositis is selected from nitrogen metabolism, Toll-like receptor signaling, NF-κB signaling, B-cell receptor signaling, P13K/AKT signaling, the cell cycle G2/M DNA damage checkpoint receptor, p38 MAPK signaling, Wnt/B-catenin signaling, glutamate receptor signaling, Integrin signaling, VEGF signaling, IL-6 signaling, Death receptor signaling, SAPK/JNK signaling, and ceramide pathway.

6. A method for modulating a molecular target involved in the onset and/or progression of oral mucositis in a patient comprising, administering to the patient a regimen of γ-D-glutamyl-L-tryptophan (SCV-07), the regimen being effective for modulating the molecular target involved in the onset and/or progression of oral mucositis.

7. The method of claim 6, wherein the molecular target is selected from nuclear factor-kappa B (NF-κB), NRF2, SP1-related retinoblastoma control protein, p53, interleukin (IL)1-β, tumor necrosis factor (TNF), and interleukin-6 (IL-6).

8. The method of claim 1, 2, 4, or 6, wherein a dose of SCV-07 is administered at least on each day of radiation treatment and/or chemotherapy.

9. The method of claim 8, wherein the dose of SCV-07 is at least about 0.02 mg/kg.

10. The method of claim 8, wherein the dose of SCV-07 is at least about 0.1 mg/kg.

11. The method of claim 8, wherein the dose of SCV-07 is at least about 0.3 mg/kg

12. The method of claim 8, wherein the dose of SCV-07 is at least about 1.0 mg/kg.

13. The method of claim 8, wherein the dose of SCV-07 is from about 0.1 mg/kg to about 2 mg/kg.

14. The method of claim 8, wherein the dose of SCV-07 is about 0.1 mg/kg.

15. The method of claim 8, wherein the dose of SCV-07 is about 0.3 mg/kg.

16. The method of claim 8, wherein the dose of SCV-07 is about 1 mg/kg.

17. The method of claim 8, wherein the dose of SCV-07 is from about 1 mg to about 100 mg.

18. The method of claim 1, 2, 4, or 6, wherein the SCV-07 is administered orally, nasally, transdermally, sublingually, by infusion, by intramuscular injection, or by subcutaneous injection.

19. The method of claim 17, wherein the dose of SCV-07 is administered by subcutaneous injection.

20. The method of claim 1, 2, 4, or 6, wherein the patient has head and neck cancer.

21. The method of claim 20, wherein the cancer is a non-metastatic squamous cell carcinoma of the oral cavity, oropharynx, hypopharynx, or larynx.

22. The method of claim 20, wherein the cancer is a salivary gland tumor, lymphoma, or sarcoma.

23. The method of claim 20, wherein the cancer is Stage III or IV.

24. The method of claim 1, 2, 4, or 6, wherein the patient has a cancer selected from breast cancer, lung cancer, ovarian cancer, colorectal cancer, or a hematologic malignancy.

25. The method of claim 20, wherein the course of radiation therapy is from 3 to 10 weeks in duration.

26. The method of claim 25, wherein the course of radiation therapy is about five to about seven weeks in duration.

27. The method of claim 25, wherein the course of radiation therapy involves radation treatment from about 5 to about 20 times per week.

28. The method of claim 26, wherein the course of radiation therapy involves radiation treatment from about 10 to about 15 times per week.

29. The method of claim 26, wherein the patient receives radiation therapy once or twice per day, at least 5 days per week.

30. The method of claim 1, 2, 4, or 6, wherein the radation therapy is Intensity-Modulated Radiation Therapy (IMRT) or External Beam Therapy (EBT).

31. The method of claim 25, wherein the course of radiation therapy is a cumulative dose of at least 30 Gy to at least one oral site.

32. The method of claim 31, wherein the course of radiation therapy is a cumulative dose of at least 40 Gy to at least one oral site.

33. The method of claim 31, wherein the course of radiation therapy is a cumulative dose of at least 50 Gy to at least one oral site.

34. The method of claim 31, wherein the course of radiation therapy is a cumulative dose of from about 50 to about 75 Gy to at least one oral site.

35. The method of claim 31, wherein a single daily fraction is from about 1.5 Gy to about 2.5 Gy to at least one oral site.

36. The method of claim 25, wherein the radiation therapy is directed to two or more 2 oral sites.

37. The method of claim 1, 2, 4, or 6, wherein the patient is receiving both chemotherapy and radiation therapy.

38. The method of claim 37, wherein the chemotherapy is cisplatin, fluorouracil, carboplatin, and/or paclitaxel.

39. The method of claim 37, wherein the chemotherapy comprises cisplatin.

40. The method of claim 39, wherein the patient receives the cisplatin at about 80 to about 100 mg/m2 from two to about four times per month.

41. The method of claim 39, wherein the patient receives cisplatin therapy from 1 to 4 times per month.

42. The method of claim 1, 2, 4, or 6, wherein the patient does not have an oral mucositis score of WHO scale 2 to 4 during at least the first 3 weeks of radiation therapy.

43. The method of claim 42, wherein the patient does not have an oral mucositis score of WHO scale 2 to 4 during at least the first 5 weeks of radiation therapy.

44. The method of claim 42, wherein the patient does not have an oral mucositis score of WHO scale 2 to 4 during the course of radiation therapy.

45. The method of claim 1, 2, 4, or 6, wherein the patient does not have an oral mucositis score of WHO scale 3 to 4 during at least the first 3 weeks of radiation therapy.

46. The method of claim 45, wherein the patient does not have an oral mucositis score of WHO scale 3 to 4 during at least the first 5 weeks of radiation therapy.

47. The method of claim 45, wherein the patient does not have an oral mucositis score of WHO scale 3 to 4 during the course of radiation therapy.

48. The method of claim 1, 2, 4, or 6, wherein the patient does not use a gastrostomy feeding tube during at least the first 4 weeks of radiation therapy.

49. The method of claim 48, wherein the patient does not use a gastrostomy feeding tube during at least the first 5 weeks of radiation therapy.

50. The method of claim 48, wherein the patient does not use a gastrostomy feeding tube during the course of radiation therapy.

51. The method of claim 1, 2, 4, or 6, wherein the patient is not administered an opioid during at least the first 3 weeks of radiation therapy.

52. The method of claim 51, wherein the patient is not administered an opioid during at least the first 5 weeks of radiation therapy.

53. The method of claim 45, wherein the patient is not administered an opioid during the course of radiation therapy.

54. The method of claim 1, 2, 4, or 6, wherein the planned course of radiation therapy is not disrupted due to oral mucositis.
